(19) United States
(12) Patent Application Publication
(10) Pub. No.: US 2022/0267843 A1
(43) Pub. Date: Aug. 25, 2022

(54) ON-CHIP OPTICAL REAL-TIME DNA SEQUENCING

(71) Applicant: Seagate Technology LLC, Fremont, CA (US)

(72) Inventor: Vivek KRISHNAMURTHY, Edina, MN (US)

(21) Appl. No.: 17/677,713

(22) Filed: Feb. 22, 2022

Related U.S. Application Data
(60) Provisional application No. 63/152,434, filed on Feb. 23, 2021.

(51) Int. Cl. C12Q 1/6872; C12Q 1/48; G01N 21/64

(52) U.S. Cl. CPC C12Q 1/6872; C12Q 1/485; G01N 21/6428; G01N 21/6408; G01N 21/6402; G01N 2201/06113; G01N 2021/6439

(57) ABSTRACT

An integrated on-chip system and methods for real-time molecular sequencing. The system has a semiconductor chip and a laser. The semiconductor chip has integrated therein a main waveguide, a plurality of branch waveguides optically connected to the main waveguide, a plurality of nanochannels each having a fluid inlet and a fluid outlet, a plurality of molecule traps, a molecule trap at an intersection of a branch waveguide and a nanochannel, and a plurality of photodetectors operably connected to the plurality of molecule traps, one photodetector for a molecule trap, the photodetector to detect a spectral signature from a molecule in the molecule trap. The laser is optically connected to the main waveguide.

### CROSS REFERENCE

This application claims priority to U.S. provisional application 63/152,434 filed Feb. 23, 2021 and titled On-Chip Optical Real-Time DNA Sequencing, the entire disclosure of which is incorporated herein by reference for all purpose.

### BACKGROUND

Current DNA sequencing methods face limitations in sequence read length, sensitivity, run time, and cost. A higher sensitivity or signal/noise ratio would improve sequencing accuracy in long reads. The length of the DNA strand to be sequenced is limited when labels are used, as most labels do not give a sufficiently strong signal and require multiple molecules to generate signals simultaneously. As the sequence length increases, the individual molecular signals fall out of sync, limiting the length of accurate sequence. Run times are often long due to the need to pause after each base incorporation to obtain an optical signal and/or remove the labels. Cost is high due to needing labels and bulky optical tools for the sequencing.

### SUMMARY

The present disclosure provides various integrated optical chip embodiments that enable laser and photodetector integration for reducing the cost, while also providing optical field enhancement for sequencing, via both labeled and label-free techniques, all in real-time.

This disclosure provides, in one particular implementation, an integrated on-chip system for real-time molecular sequencing. The system comprises a semiconductor chip and a laser. The semiconductor chip has integrated therein a main waveguide, a plurality of branch waveguides optically connected to the main waveguide, a plurality of nanochannels each having a fluid inlet and a fluid outlet, a plurality of molecule traps, a molecule trap at an intersection of a branch waveguide and a nanochannel, and a plurality of photodetectors operably connected to the plurality of molecule traps, one photodetector for a molecule trap, the photodetector to detect a spectral signature from a molecule in the molecule trap. The laser is optically connected to the main waveguide.

This disclosure also provides, in another particular implementation, another integrated on-chip system for real-time molecular sequencing. The system comprises a semiconductor chip, a laser, and a computing system. The semiconductor chip has integrated therein a plurality of waveguides, a plurality of nanochannels intersecting the plurality of waveguides, a molecule trap at an intersection of a waveguide and a nanochannel, and a photodetector operably connected to the molecule trap, the photodetector to detect a spectral signature from a molecule in the molecule trap. The laser is optically connected to the main waveguide. The computing system is operably connected to the plurality of photodetectors to identify the spectral signature.

Still also, this disclosure provides, in another particular implementation, a method for sequencing DNA in real-time. The method includes providing a DNA single strand to a molecule trap at an intersection of a waveguide and a nanochannel, the waveguide and the nanochannel integrated in a semiconductor chip. The method also includes providing a light source from a laser to the molecule trap via the waveguide. The method further includes monitoring a spectral signature via a photodetector operably connected to the molecule trap, the spectral signature indicative of a free nucleotide attaching to the DNA single strand, and correlating the spectral signature to a nucleotide.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. These and various other features and advantages will be apparent from a reading of the following detailed description.

### BRIEF DESCRIPTION OF THE DRAWING

The described technology is best understood from the following Detailed Description describing various implementations read in connection with the accompanying drawing, where:

FIG. 1 is a tabular representation of four nucleotides and possible signature signals.

FIG. 2A is a schematic top view of an integrated sequencing system; FIG. 2B is an enlarged view of a portion of FIG. 2A, showing one sensor; FIG. 2C is a cross-sectional side view taken along line C-C of FIG. 2B; and FIG. 2D is a cross-sectional side view taken along line D-D of FIG. 2B.

FIG. 3A is a schematic top view of another integrated sequencing system, with a spectrometer; FIG. 3B is an enlarged view of a portion of FIG. 3A, showing one sensor; FIG. 3C is a cross-sectional side view taken along line C-C of FIG. 3B; and FIG. 3D is a cross-sectional side view taken along line D-D of FIG. 3B.

FIG. 4A is an enlarged view of a portion of an integrated sequencing system, similar to FIG. 2B; FIG. 4B is a cross-sectional side view taken along line B-B of FIG. 4A; FIG. 4C is an enlarged view of a portion of another integrated sequencing system; and FIG. 4D is a cross-sectional side view taken along line D-D of FIG. 4C.

FIG. 5A is a schematic view of single molecule real-time sequencing in a molecule trap utilizing a first methodology; FIG. 5B is a schematic view of single molecule real-time sequencing in a molecule trap utilizing a second methodology.

FIG. 6A is a schematic cross-sectional side view of a sensor of an integrated sequencing system; FIG. 6B is a graphical representation of fluorescence intensity and colors over time; FIG. 6C is a graphical representation of fluorescence intensity and time decay, over time.

FIG. 7A is a schematic top view of a portion of an integrated sequencing system; FIG. 7B is a graphical representation of Raman spectra for nucleotides A, C, G, T.

FIG. 8A is a schematic top view of a laser coupling to an integrated chip; FIG. 8B is a schematic side view of another laser coupling to an integrated chip.

FIG. 9 is a block diagram of a computing system suitable for use with the systems described herein.

### DETAILED DESCRIPTION

As indicated above, the methods described herein provide real-time DNA sequencing via an optical system, that can be used with DNA strands, either labeled or label-free.

Many commercial optical sequencing methods utilize labeled (or tagged) nucleotides for SERS and other Raman spectroscopy. Most methods require expensive and bulky optical instruments, to couple the light from a laser and to detect the Raman signature from the sample area. Typically, this equipment includes a laser source, a detector, and a separate zero waveguide chip, which cannot be integrated. Hence, the methods depend on the instrumentation from that company.

Provided herein are methods and systems that integrate a laser, a coupler, waveguides, and a photodetector that leverages CMOS foundry processes, to bypass the need for bulky optical instruments and to provide a low-cost and portable solution for DNA, RNA and protein sequencing. The methods can use labeled or label-free molecules. Complete on-chip integration makes it compact and low cost and thus having a much wider commercialization possibility as a portable system for DNA, RNA and protein sequencing.

The methods and systems of this disclosure utilize a CMOS chip having at least one molecular trap (e.g., DNA trap) as a sample chamber, the trap optically connected to a laser light source via a waveguide that can include a coupler, polarization rotator, etc. Each trap is operable connected to a photodetector to detect a nucleotide's spectral signature. The spectral signature signal may be enhanced by resonance of focusing plasmonic (e.g., gold, silver) nanostructures. The nanostructures provide focusing points, e.g., on either side of the channel or waveguide through which the DNA strand passes during sequencing. The chip may or may not be integrated with a spectrometer.

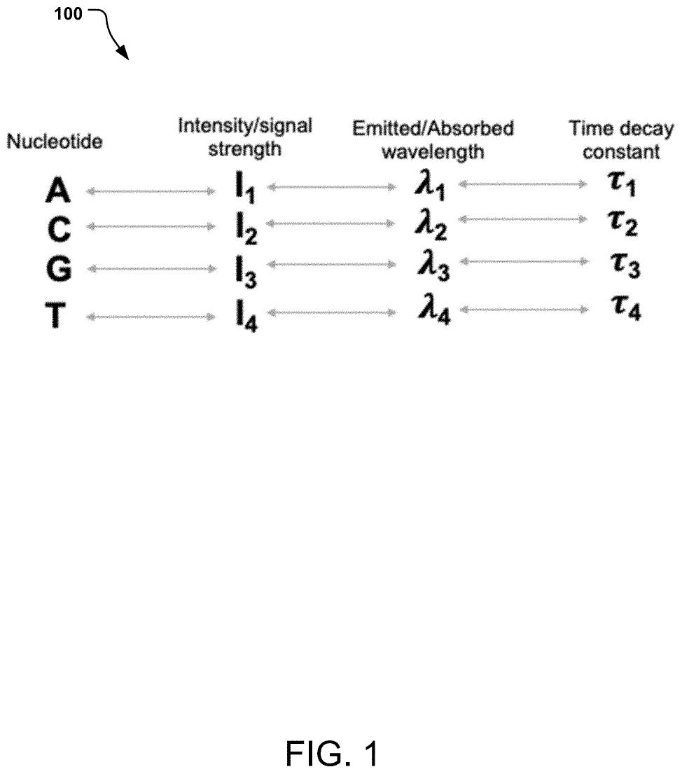

General approach to DNA, RNA and protein optical sequencing of this disclosure involves providing the molecule to be sequenced to a sample area (referred to as a trap herein) where the electromagnetic field is concentrated or enhanced, e.g., in a “hot spot” formed by laser excitation and enhanced by resonance of focusing plasmonic (e.g., gold, silver) nanostructures, with the purpose of maximizing energy input into the sample molecule. The sample molecule emits energy that has some nucleotide-specific signature. FIG. 1 provides a table listing the four nucleotides adenine (A), cytosine (C), guanine (G), and thymine (T) and their signature; the signature can be in signal intensity domain, wavelength domain, or time domain. A label or tab may facilitate distinguishing nucleotides in one or more of these output signatures. The following discussion provides various integrated sequencing systems that utilize these output signatures to identify the nucleotides and their order.

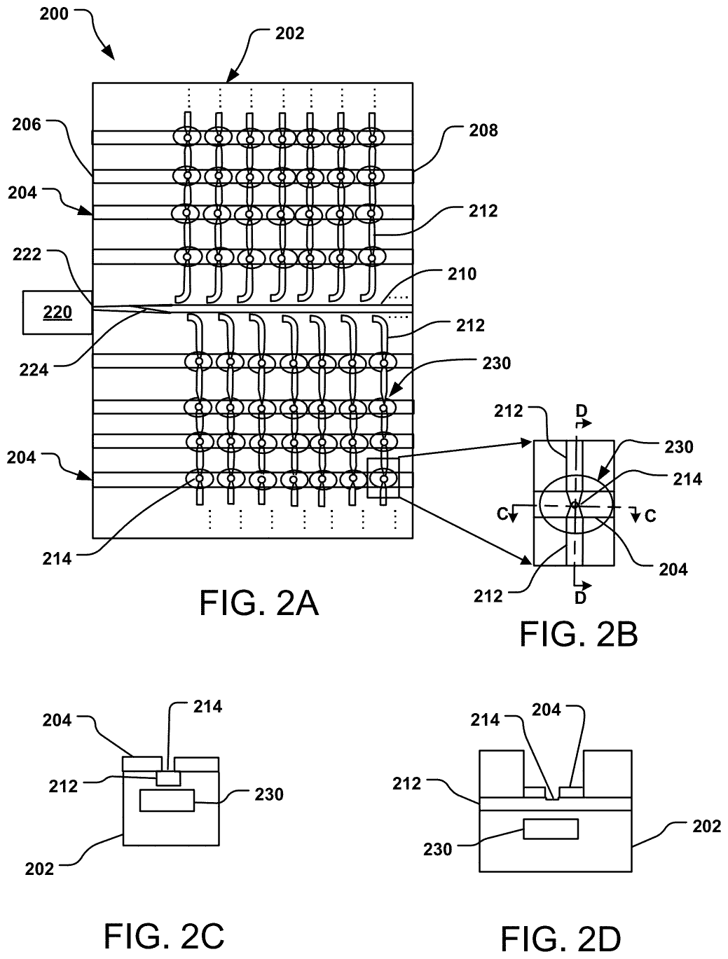

FIG. 2A shows a system 200 for sequencing that does not include a spectrometer with an integrated chip.

The system 200 includes CMOS chip 202 (e.g., formed of silicon, e.g., fused silicon) with a plurality of nanochannels 204 extending therein, each nanochannel 204 having a fluid inlet 206 and a fluid outlet 208; these nanochannels 204 are fluidly connected at their inlet 206 to a source of the molecule(s) to be sequenced, the molecule(s) being in a fluid (e.g., a liquid). In this particular embodiment of system 200 there are eight nanochannels 204; in other embodiments, there may be, e.g., as many as one hundred nanochannels 204 on a chip 202. In some embodiments, the nanochannels 204 are approximately 100 nanometers thick, and formed of Al, Au, or other chemically inert metal. The nanochannels 204 are physically formed in the chip 202 by removal of a portion of the chip 202 and may be channels, having an open top side (e.g., similar to a trough), or may be fully enclosed passages. In some embodiments, the nanochannels 204 have a depth and/or a width of approximately 1 micrometer.

Integrated into the CMOS chip 202 is a main waveguide 210 that is optically coupled to a laser 220 external to the chip 202; in some embodiments, more than one laser 220 may be used. The main waveguide 210 extends from an outer edge of the chip 202 into and optionally to an opposite outer edge of the chip 202. The main waveguide 210 may be formed in the chip 202 by, e.g., removal of a portion of the chip 202, may be formed in the chip 202 with a previously formed waveguide, or may be formed on the surface of the chip 202 by, e.g., positioning of a previously formed waveguide. One example of a suitable main waveguide is approximately 100-300 nanometer thick and formed of silicon nitride or a material that is transparent at visible wavelengths.

The laser 220 can be physically coupled to the edge of the chip 202 at the main waveguide 210 with an optical coupler 222. A polarization rotator 224 may be present in the main waveguide 210 to correct polarization of an electromagnetic beam for more efficient interaction with the sequencing area. An example of a polarization rotator 224 is an IBEX-like polarization rotator may be used.

Optically coupled to the main waveguide 210, in this design extending orthogonal to the main waveguide 210, are a plurality of branch waveguides 212, which are coupled via branch coupler (not called out) to affect the input power (light) entering each branch waveguide 212. The waveguides 212 may be, e.g., approximately 100-300 nanometer thick and formed of, for example, silicon nitride.

At the intersection of a nanochannel 204 with a waveguide 212 is a molecule trap 214; if DNA is being sequenced, this may be called a DNA trap 214. If the molecule being sequenced is DNA, the trap 214 can include a DNA polymerase present, e.g., bound to a surface of the trap 214. In some embodiments, a surface of the trap 214 may be activated, e.g., using PVPA (polyvinylphosphonic acid) to increase the likelihood of the polymerase and the DNA being immobilized on a surface of the trap 2145, such as at the bottom of the trap 214. In some embodiments, the trap 214 has a width or diameter of approximately 20-150 nanometers.

Positioned in close proximity to the trap 214 is a photodetector 230 (e.g., silicon photodetector). The photodetector 230 is positioned below the nanochannel 204, the waveguide 212 and the trap 214, integrated into the chip 202. In this embodiment, the photodetector 230 is shown having a circular or oval shape having a diameter of about 5 to 20 micrometers. The photodetector 230 could be patterned for improving photodetector response. The photodetector 230 is operably connected to appropriate measurement and/or computational devices to monitor and determine the spectral signature in the trap 230. Together, the trap 214 and the photodetector 230 may be referred to as a sensor.

The system 200 has four sensors on each of seven branch waveguides 214 on each side of the main waveguide 210, providing a total of 56 sensors in the shown system 200. In other embodiments, there can be more than 100 sensors or more than 200 sensors on the chip 202, e.g., in the system 200. The number of sensors may be increased by increasing the number of branch waveguides than shown in FIG. 2A and/or by increasing the number of nanochannels intersecting with the branch waveguides. By having multiple sensors running simultaneously, the accuracy and/or rate of sequencing is increased. The overall width of the system 200 and/or the chip 202 can be, e.g., 10 to 100 mm, depending on the number of sensors.

FIG. 2B shows an enlargement of the sensor (that is, at least the trap 214 and the photodetector 230) from FIG. 2A at an intersection of a nanochannel 204 and waveguide 212. FIG. 2C shows a side view of the sensor taken along line C-C of FIG. 2B and FIG. 2D shows a side view of the sensor taken along line D-D of FIG. 2B.

As seen in FIG. 2B, the top view, the waveguide 212 tapers proximate the trap 214, to a waveguide tip 215, in some embodiments to a width of about 20150 nanometers. The relative orientation of the waveguide 212, the nanochannel 204, the trap 214, and the photodetector 230 is seen in the side views of FIGS. 2C and 2D.

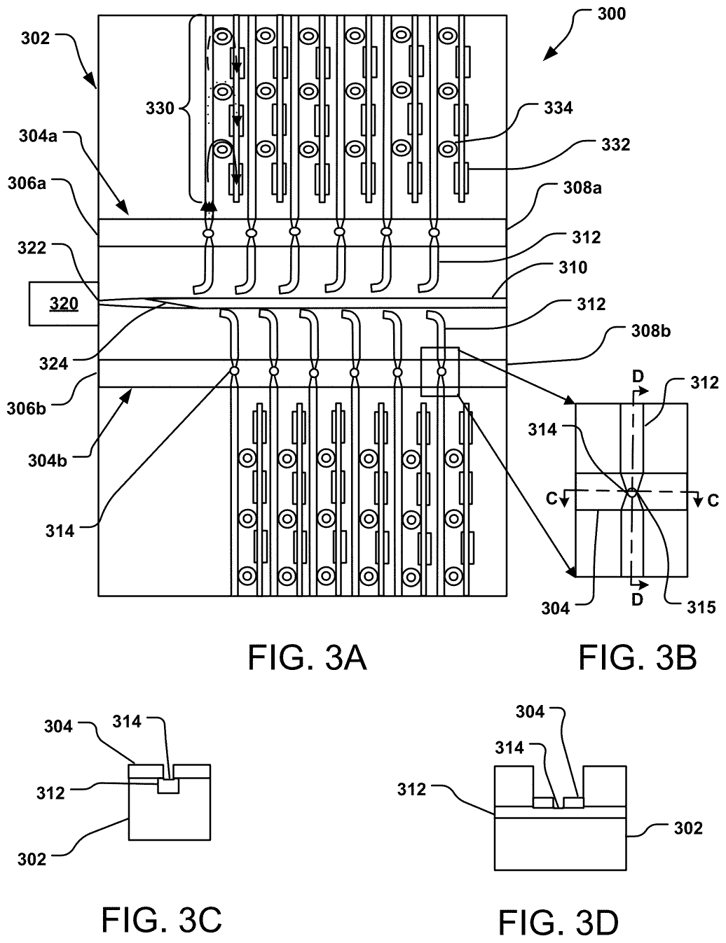

FIG. 3A shows another integrated system 300 for sequencing, which includes a spectrometer with the integrated chip. It is to be understood that various features and/or details from the system 200, described above, may be applied to this system 300 unless contrary to the construction or the process of the integrated system.

The system 300 includes CMOS chip 302 with two nanochannels 304a, 304b extending therein, each nanochannel 304a, 304b having an inlet 306a, 306b and an outlet 308a, 308b, respectively. These nanochannels 304 are fluidly connected at their inlet 306 to a source of the molecule(s) to be sequenced. If the molecule being sequenced is DNA, the nanochannels 304 can include a DNA polymerase, e.g., bound to a surface of the nanochannel 304. In some embodiments, the nanochannels 304 have a depth and/or a width of approximately 1 micrometer.

Integrated into the CMOS chip 302, extending between the two nanochannels 304, is a main waveguide 310 that is optically coupled to a laser 320 external to the chip 302. The main waveguide 310 extends from an outer edge of the chip 302 into and optionally to an opposite outer edge of the chip 302.

The laser 320 can be physically coupled to the edge of the chip 302 at the main waveguide 310 with an optical coupler 322. A polarization rotator 324 may be present to correct polarization of an electromagnetic beam for more efficient interaction with the sequencing area.

Optically coupled to the main waveguide 310, in this design extending orthogonal to the main waveguide 310, are a plurality of branch waveguides 312. A branch coupler (not called out) can be included to affect the input power (light) entering each branch waveguide 312.

At the intersection of the nanochannel 304 with a waveguide 312 is a molecule trap 314. If the molecule being sequenced is DNA, the trap 314 can include a DNA polymerase, e.g., bound to a surface of the trap 314. FIG. 3B shows an enlargement of a trap 314 from FIG. 3A at an intersection of a nanochannel 304 and waveguide 312. FIG. 3C shows a side view of the trap 314 taken along line C-C of FIG. 3B and FIG. 3D shows a side view of the trap 314 taken along line D-D of FIG. 3B.

As seen in FIG. 3B, the top view, the waveguide 312 tapers at approximately the center of the trap 314, to a waveguide tip 315, in some embodiments having a width of about 20-150 nanometers. The relative orientation of the waveguide 312, the nanochannel 304, and the trap 314 is seen in FIGS. 3C and 3D. Like in the system 200 of FIGS. 2A through 2D, the trap 314 is merely an area in the nanochannel 304 devoid of material; that is, the trap 314 is a void in the nanochannel 304 extending through to the waveguide 312.

Unlike in the system 200, each waveguide 312 intersects only one nanochannel 304 and thus powers only one trap 314. Optically connected to each waveguide 312 is a spectrometer 330, integrated with the chip 302. The spectrometer 330 includes photodetectors 330 (e.g., silicon photodetectors) under the waveguide 312; in some embodiments, the photodetectors 332 are elongate along the waveguide 312, having a length of approximately 50-100 micrometers. The spectrometer 330 also includes ring resonator filters 334, one for each photodetector 332; in this design, the spectrometer 330 has three photodetectors 332 and three ring resonator filters 334 for each waveguide 312 and each trap 314. The ring resonator filter 334 selects the optical field (e.g., at a given wavelength) and limits the power passing to the photodetector 332 to that wavelength. In some embodiments, a filter other than a ring resonator filter may be used; examples of suitable filters include diffraction gratings, prisms, edge filters, notch filters, bandpass filters, directional couplers, MZI (Mach-Zehnder Interferometer) filters, AWG (Array waveguide gratings), etc.

In operation, light from the laser 320 enters the main waveguide 304 and is coupled to the branch waveguides 312, to interact with the molecule (e.g., DNA) in the trap 314. A specific spectral signature (e.g., wavelength) of light is emitted from the molecule. For DNA, the signature emitted is based on the complementary nucleotide being added to the single strand DNA by the polymerase. The emitted signature light is filtered and channeled to the appropriate photodetector 332 by the ring resonator filter 334.

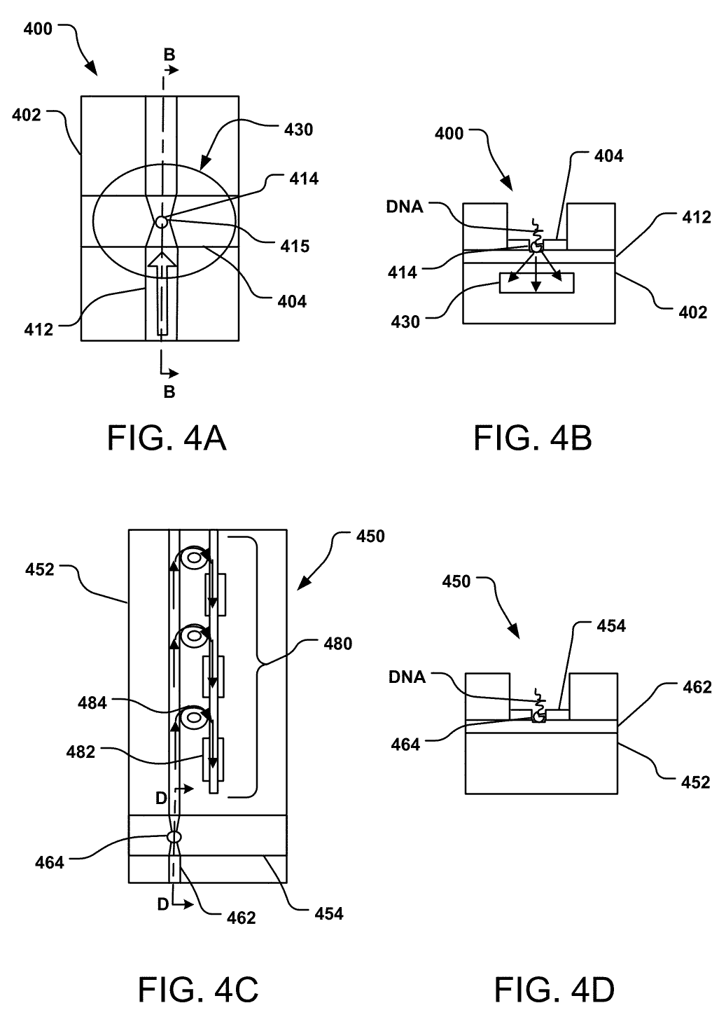

FIGS. 4A and 4B schematically illustrate additional details regarding a sensor similar to one from the system 200 of FIGS. 2A through 2D and FIGS. 4C and 4D schematically illustrate additional details regarding a sensor similar to one from the system 300 of FIGS. 3A through 3D.

FIGS. 4A and 4B show a sensor 400 on a chip 402; the sensor 400 is one of possibly hundreds of sensors on the chip 402 forming a system. The sensor 400 has a nanochannel 404 intersecting a waveguide 412 forming a trap 414. A photodetector 430 is positioned proximate the trap 414 and the nanochannel 404 and the waveguide 412. In FIG. 4A, the light from the laser, which also can be referred to as an optical field, is shown propagating along the waveguide 412 by the arrow to the trap 414. The light in the waveguide 412 interacts with a molecule (e.g., DNA single strand) present in the trap 414.

A single-strand DNA is shown in the trap 414 in FIG. 4B. A DNA polymerase is present in the trap 414, as well as free (unattached) nucleotides (e.g., A, C, T, G), which are not seen. Although not necessary, the free nucleotides can be labeled or tagged. The DNA strand is captured by the polymerase and is primed for transcription. The appropriate complementary free nucleotide is paired to the template strand, one at a time.

The incorporation of the free nucleotide results in the emittance of a signal depending on which nucleotide (adenine (A), cytosine (C), guanine (G), and thymine (T)) is incorporated to the strand. The photodetector 430 detects the output signal, such as a fluorescence signal (e.g., if labeled) or Raman signal, in the form of intensity, wavelength (color) and/or time decay. Based on the detected signal, as the DNA flows continuously, a specific pattern of DNA sequence, associated with the specimen molecule, is generated.

FIGS. 4C and 4D show a sensor 450 on a chip 452, where the trap is coupled to a photodetector through a spectrometer; the sensor 450 is one of possibly hundreds of sensors on the chip 452 forming a system. The sensor 450 has a nanochannel 454 intersecting a waveguide 452 forming a trap 464. In this sensor 450, the signal generated at the trap 464 is coupled to an outgoing waveguide that carries the signal to a spectrometer 480, which in this embodiment has three photodetectors 482 and three ring resonator filters 484. The direction of the light from the laser is indicated by the arrows. In other embodiments, a different mechanism may be used for filtering or separating the signal, such as diffraction gratings, prisms, edge filters, notch filters, bandpass filters, directional couplers, MZI (Mach-Zehnder Interferometer) filters, AWG (Array waveguide gratings), etc.

A single-strand DNA is shown in the trap 464 in FIG. 4D. A DNA polymerase is present in the trap 464, as well as free (unattached) nucleotides (e.g., A, C, T, G) which are not seen in the figure. The DNA strand is captured by the polymerase and is primed for transcription. The appropriate complementary free nucleotide is paired to the template strand, one at a time.

The light in the waveguide 452 interacts with the DNA strand present in the trap 464. The incorporation of the free nucleotide results in the emittance of a signal depending on which nucleotide (A, C, T, G) is incorporated to the strand. Depending on the specific signal, the ring resonator 484 or other filter guides the signal to a specific photodetector 482 or enables a specific response of the detector, thus identifying the intensity or wavelength of the signal and correlating the signal to a particular nucleotide. Based on the detected signal, as the DNA flows continuously, a specific pattern of nucleotide sequence is generated.

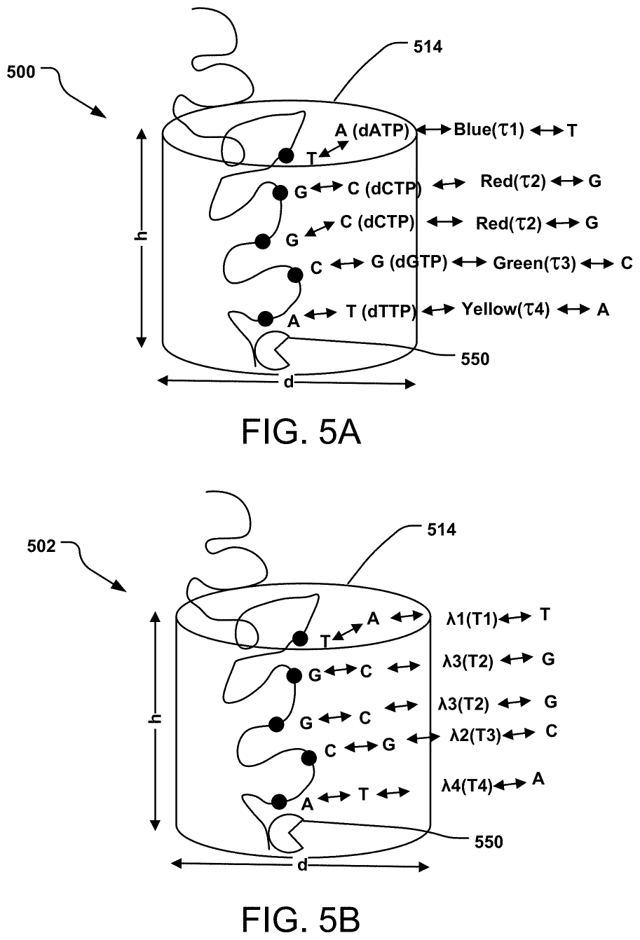

FIGS. 5A and 5B schematically show two methodologies of single molecule real-time sequencing in an integrated system, such as the system 200 or the system 300. FIG. 5A shows a methodology 500 utilizing fluorescence labeled or tagged free nucleotides and FIG. 5B shows a methodology 502 using untagged free nucleotides. In both figures, the methodologies utilize a trap 514.

The trap 514, similar to either the trap 214 of the system 200 or the trap 314 of the system 300, is a molecule trap, e.g., located at an intersection of a fluid nanochannel and a light source, such as a waveguide. The trap 514 has a volume, in this embodiment where the trap 514 is cylindrical, defined by a height h and a width or diameter w. In some embodiments, the height is about 100 nanometers and the width is about 20-150 nanometers, e.g., about 70 nanometers.

Present within the volume of the trap 514 is a DNA polymerase 550, optionally bounded to a wall or the floor of the trap 514. PVPA (polyvinylphosphonic acid) may be present to facilitate adsorption of the polymerase/DNA to the bottom of the trap, for example, the PVPA enables adsorption to the fused silica surface, creating a small observation window (e.g., about 70-100 nm wide holes).

Also present within the trap 514 are free nucleotides or oligonucleotides (e.g., A, C, G, T) and a complimentary primer. The nucleotides may or may not be labeled, e.g., with fluorescence. Both figures show a DNA single strand in the trap 514, with nucleotides A, C, G, T identified on the strand. The complimentary free nucleotide for A is T; the complimentary free nucleotide for C is G; the complimentary free nucleotide for G is C (twice); and the complimentary free nucleotide for T is A. A complementary nucleotide is attached to the DNA single strand, in order, approximately every 1 millisecond to a second, depending on the specific polymerase used.

FIG. 5A has fluorescence labeled or tagged free nucleotides present in the trap 514, shown as A(dATP), C(dCTP) twice, G(dGTP) and T(dTTP). As the free nucleotides incorporate into the single DNA strand, a signal is emitted depending on which nucleotide (A(dATP), C(dCTP), G(dGTP), T(dTTP)) is incorporated to the strand, the signal varying by, e.g., intensity of fluorescence, color (wavelength) of fluorescence, or time decay constant (τ1, τ2, τ3, τ4). For example, referring to FIG. 5A, a blue wavelength or first decay constant (τ1) represents the A nucleotide being added to the strand and thus the original nucleotide being T, a red wavelength or second decay constant (τ2) represents the C nucleotide being added to the strand and thus the original nucleotide being G, a green wavelength or third decay constant (τ3) represents the G nucleotide being added to the strand and thus the original nucleotide being C, and a yellow wavelength or fourth decay constant (τ4) represents the T nucleotide being added to the strand and thus the original nucleotide being A. From the changing signal, the sequence of nucleotides can be determined.

FIG. 5B has non-labeled or untagged free nucleotides present in the trap 514. As the free nucleotides incorporate into the single DNA strand, a signal is emitted depending on which nucleotide (A, C, G, T) is incorporated to the strand, the signal varying by, e.g., intensity, wavelength (λ1, λ2, λ3, λ4) or time decay constant (T1, T2, 13, T4) of the Raman signal emitted. For example, referring to FIG. 5B, a first wavelength (λ1) or first decay constant (T1) represents the A nucleotide being added to the strand and thus the original nucleotide being T, a second wavelength (λ2) or second decay constant (T2) represents the C nucleotide being added to the strand and thus the original nucleotide being G, a third wavelength (λ3) or third decay constant (T3) represents the G nucleotide being added to the strand and thus the original nucleotide being C, and a fourth wavelength (λ4) or fourth decay constant (T4) represents the T nucleotide being added to the strand and thus the original nucleotide being A. From the changing signal, the sequence of nucleotides can be determined.

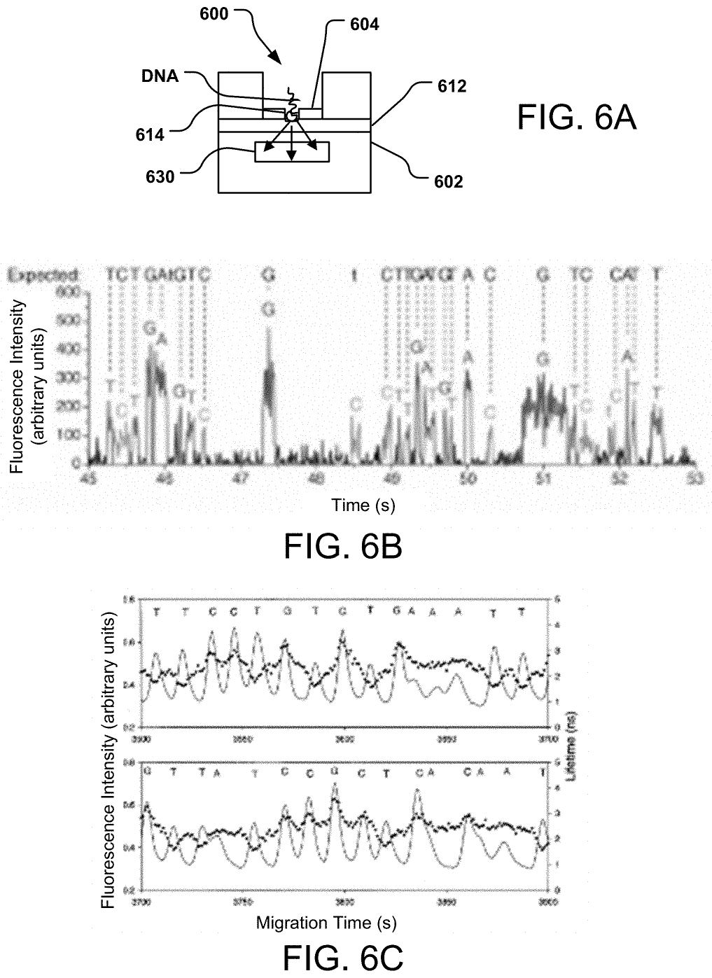

FIGS. 6A, 6B and 6C provide examples directed to utilizing fluorescence labeled free nucleotides and how the outgoing wavelength or time decay constant can be used to identify nucleotides.

FIG. 6A shows a sensor 600 similar to the sensor 400 of FIG. 4B. The sensor 600 is formed on a chip 602 and has a nanochannel 604 intersecting a waveguide 612 forming a trap 614 formed in the chip 602; a DNA polymerase is present in the trap 614, as well as fluorescence labeled or tagged free nucleotides, which are not seen. A photodetector 630 is positioned proximate the trap 614. Light from a laser (not shown) interacts with a molecule (e.g., DNA single strand) present in the trap 614.

The incorporation of the free nucleotide results in the emittance of a signal depending on which nucleotide (A, C, T, G) is incorporated to the strand. The photodetector 630 detects the output signal emitted depending on which nucleotide is incorporated to the strand, the signal varying by, e.g., intensity of fluorescence, color (wavelength) of fluorescence, or time decay constant of fluorescence.

FIG. 6B shows an example graphical representation of fluorescence intensity, over time, showing the different intensity values for different nucleotides and thus identifying the nucleotides.

FIG. 6C shows two example graphical representations of fluorescence intensity and fluorescence time decay, over time, showing the different intensity values for different nucleotides and thus identifying the nucleotides.

Details regarding various methodologies for fluorescence time decay can be found in, e.g., “Development of a Time Domain Fluorimeter for Fluorescent Lifetime Multiplexing Analysis” by Christopher D. Salthouse et al., IEEE Transactions on BioMedical Circuits and Systems, Vol. 2, No. 3, September 2008, and “DNA Sequencing by Capillary Electrophoresis with Four-Decay Fluorescence Detection” by Hui He et al. from the Department of Chemistry at Duke University, Analytical Chemistry, Vol. 72, pp. 5865-5873, 2000. See also, “Bright Unidirectional Fluorescence Emission of Molecules in a Nanoaperture with Plasmonic Corrugations” by Heykel Aouani et al., Wiley Interdisciplinary Reviews Nanomedicine and Nanobiotechnology, May 2014.

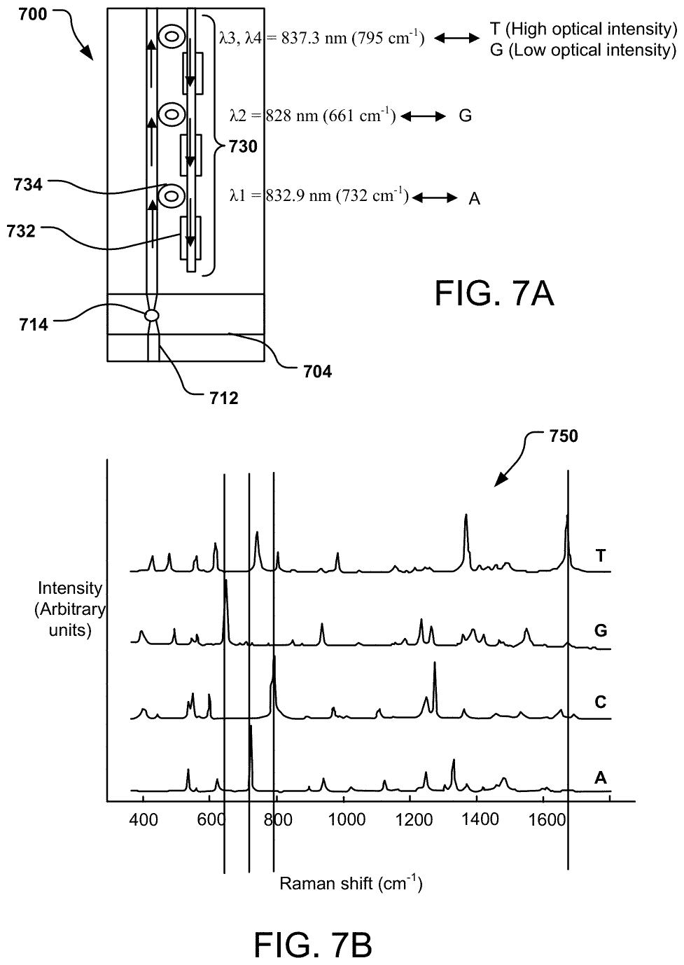

FIG. 7A shows a sensor 700, similar to the sensor 450 shown in FIG. 4C, particularly, having a single waveguide, molecule trap, and spectrometer. In FIG. 7A, this sensor 700 has a nanochannel 704, a waveguide 712, a molecule trap 714 at the intersection of the nanochannel 704 and the waveguide 712, and a spectrometer 730 having three photodetectors 732 and three ring resonator filters 734. The direction of the light is indicated by the arrows.

The sensor 700 can be used for real-time DNA sequencing, for example, with non-labeled Surface Enhanced Raman Spectroscopy (SERS) to identify the four DNA nucleotides (A, C, G, T) based on their unique Raman scattering spectra.

In this particular embodiment, with the input laser wavelength at approximately 785 nm, the first ring resonator filter 734a is centered on a wavelength of 832.9 nm (732 cm⁻¹) which corresponds to approximately the A nucleotide, the second ring resonator filter 734b is centered on a wavelength of 828 nm (661 cm⁻¹) which corresponds to approximately the G nucleotide, and the third ring resonator filter 734c is centered on a wavelength of 837.3 nm (795 cm⁻¹), approximately corresponds to the spectral signature from the T nucleotide if the high optical intensity signal is detected and the G nucleotide if the low optical intensity signal is detected.

FIG. 7B shows a graph 750 of the Raman spectra of nucleotides adenine (A), cytosine (C), guanine (G), and thymine (T) at an excitation wavelength of approximately 514.5 nm. Example peaks that may be used for nucleotide identification are identified in FIG. 7B: 721 cm⁻¹ for A, 776 cm⁻¹ for C, 643 cm⁻¹ for G, and 1680 cm⁻¹ for T. Thus, if the laser injection wavelength into the sensor 700 of FIG. 7A had been about 514.5 nm, the filters would be centered approximately around 721 cm⁻¹ for A, 776 cm⁻¹ for C, 643 cm⁻¹ for G, and 1680 cm⁻¹ for T, as shown in FIG. 7B.

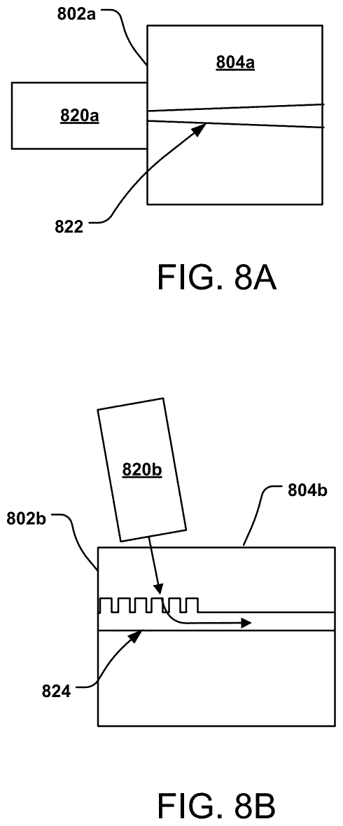

FIGS. 8A and 8B schematically show two options for integrating a laser to the chip, as in the system 200 and the system 300. Both options have a chip 802 having a side edge 802 and a top surface or edge 804. In both options, the laser can be optically coupled into a waveguide and to plasmonic nanostructures.

In FIG. 8A, a top view, a laser 820a is shown abutted to an edge of a chip 802a, the output light from the laser 820a aligned with a main waveguide via an edge coupler 822. Such an arrangement can be considered “in-plane.”

In FIG. 8B, a side view, a laser 820b is shown optically connected to a surface 804b of a chip 802b, the output light from the laser 820b aligned with a main waveguide via a grating coupler 824. Such an arrangement can be considered “out-of-plane.” Additionally, such an arrangement can be used to integrate the laser and the DNA sequencing chip in a HAMR (heat-assisted magnetic recording) drive-like module.

The laser 820 could be, for example, a pulsed laser (e.g., a mode-locked laser) or a continuous wave (CW) laser that can be modulated. The laser 820 may be physically attached to the chip (e.g., adhered, soldered, welded, clipped, or otherwise mounted), integrated into the chip, or the laser could be movable in relation to the chip. For designs where the laser 820 is attached to the chip, the chip and the coupler (e.g., the edge coupler 822 or grating coupler 824) can be placed on a fiat surface, to interact with the laser 820. Similarly, for designs where the coupler 822, 824 is integral with the chip, the laser 820 and the chip can be aligned and placed on a flat surface. As an example of a laser 820 that is movable relative to the chip, the chip can be a disk-drive media and the laser 820 can be incorporated into the head gimbal assembly (HGA) of the disk-drive. The laser 820 can be moved into relative position with the coupler 822, 824 and the main waveguide by movement of the and/or the rotating disk. For arrangements where the laser is not integrated into the chip, the laser is removable therefrom so that the chip can be disposable, e.g., to enable sequencing of various specimen.

With either system 200 or system 300, light from the laser 220, 320 couples to the main waveguide 210, 310 and to the branch waveguides 212, 312 to interact with the molecule in the trap 214, 314. A photodetector 230, 332 detects the signature signal emitted by nucleotides to identify the nucleotides.

If the molecule is DNA, each nucleotide of the DNA single strand is characterized through single molecule real-time sequencing; the sequencing may be with either labeled or not-labeled nucleotides.

The systems and sensors described herein, particular the photodetectors, are operably connected to a computing system 900 suitable for implementing the sensing and identification of the spectral signature, e.g., the wavelength, the fluorescence, etc. The computing system 900 may be, e.g, a computer (e.g., a laptop or a desktop) or a mobile device such as a mobile phone or a tablet. The computing system 900 is capable of executing a computer program product embodied in a tangible computer-readable storage medium to execute a computer process for any sensing and identification. As used herein, “tangible computer-readable storage media” includes, but is not limited to, RAM, ROM, EEPROM, flash memory or other memory technology, CDROM, digital versatile disks (DVD) or other optical disk storage, magnetic cassettes, magnetic tape, magnetic disk storage or other magnetic storage devices, or any other tangible medium that can be used to store the desired information and that can accessed by a computer. In contrast to tangible computer-readable storage media, intangible computer-readable communication signals may embody computer readable instructions, data structures, program modules or other data resident in a modulated data signal, such as a carrier wave or other signal transport mechanism. The term “modulated data signal” means a signal that has one or more of its characteristics set or changed in such a manner as to encode information in the signal.

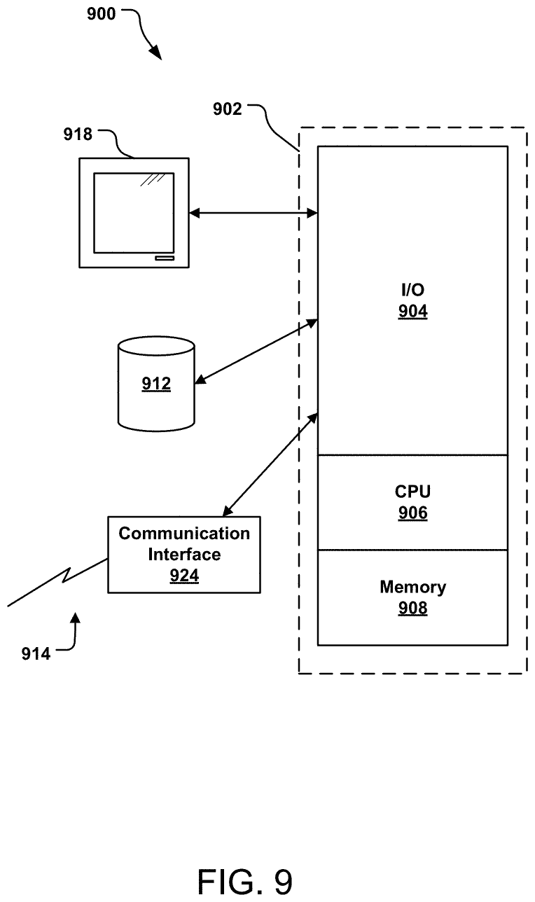

Data and program files may be input to the computing system 900, which reads the files and executes the programs using one or more processors. Some of the elements of the computing system 900 are shown in FIG. 9. The system 900 has a processor 902 having an input/output (I/O) section 904, a Central Processing Unit (CPU) 906, and a memory section 908. There may be one or more processors 902 in the system 900, such that the processor 902 of the computing system 900 comprises a single CPU 906, or a plurality of CPUs 906. The processors 902, 906 may be single core or multi-core processors.

The computing system 900 may be a conventional computer, a distributed computer, or any other type of computer. The described technology is optionally implemented in software (modules) loaded in memory 908, a storage unit 912, and/or communicated via a wired or wireless network link 914 on a carrier signal (e.g., Ethernet, 3G wireless, 5G wireless, LTE (Long Term Evolution)) thereby transforming the computing system 900 in FIG. 9 to a special purpose machine for implementing the described operations.

The I/O section 904 may be connected to one or more user-interface devices (e.g., a keyboard, a touch-screen display unit 918, etc.) or a storage unit 912. Computer program products containing mechanisms to effectuate the systems and methods in accordance with the described technology may reside in the memory section 908 or on the storage unit 912 of such a computing system 900.

A communication interface 920 is capable of connecting the computing system 900 to a network via the network link 914, through which the computing system can receive instructions and data embodied in a carrier wave. When used in local area networking (LAN) environment, the computing system 900 is connected (by wired connection or wirelessly) to a local network through the communication interface 920, which is one type of communications device. When used in a wide-area-networking (WAN) environment, the computing system 900 typically includes a modem, a network adapter, or any other type of communications device for establishing communications over the wide area network. In a networked environment, program modules depicted relative to the computing system 900 or portions thereof, may be stored in a remote memory storage device. It is appreciated that the network connections shown are examples of communications devices for and other means of establishing a communications link between the computers may be used.

One or more relational databases storing data used in comparing different measurements may be stored in the disc storage unit 912 or other storage locations accessible by the computing system 900. In addition, the computing system 900 may utilize a variety of online analytical processing tools to mine and process data from the databases. Further, local computing systems, remote data sources and/or services, and other associated logic represent firmware, hardware, and/or software, which may be configured to characterize and compare different locales. A monitoring system of this disclosure can be implemented using a general purpose computer and specialized software (such as a server executing service software), a special purpose computing system and specialized software (such as a mobile device or network appliance executing service software), or other computing configurations. In addition, any or all of the module(s) may be stored in the memory 908 and/or the storage unit 912 and executed by the processor 902.

The implementations for processing the spectral signal, described herein, can be implemented as logical steps in one or more computing systems. The logical operations of the present invention are implemented (1) as a sequence of processor-implemented steps executing in one or more computing systems and (2) as interconnected machines or circuit modules within one or more computing systems. The implementation is a matter of choice, dependent on the performance requirements of the computing system implementing the invention. Accordingly, the logical operations making up the implementations described herein are referred to variously as operations, steps, objects, or modules. Furthermore, it should be understood that logical operations may be performed in any order, adding and omitting as desired, unless explicitly claimed otherwise or a specific order is inherently necessitated by the claim language.

In summary, described herein are methods of utilizing labeled and non-labeled techniques to sequence a DNA strand (e.g., a template strand) or an RNA strand or proteins with focusing plasmonic nanostructures that create a hot spot. The systems and sensors are on-chip, and can include an immobilized DNA polymerase, which replicates the template strand being sequenced. The strand (or nucleotides) may be labeled or non-labeled.

The above specification and examples provide a complete description of the structure and use of exemplary implementations of the invention. The above description provides specific implementations. It is to be understood that other implementations are contemplated and may be made without departing from the scope or spirit of the present disclosure. The above detailed description, therefore, is not to be taken in a limiting sense. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

Spatially related terms, including but not limited to, “bottom,” “lower”, “top”, “upper”, “beneath”, “below”, “above”, “on top”, “on,” etc., if used herein, are utilized for ease of description to describe spatial relationships of an element(s) to another. Such spatially related terms encompass different orientations of the device in addition to the particular orientations depicted in the figures and described herein. For example, if a structure depicted in the figures is turned over or flipped over, portions previously described as below or beneath other elements would then be above or over those other elements.

Since many implementations of the invention can be made without departing from the spirit and scope of the invention, the invention resides in the claims hereinafter appended. Furthermore, structural features of the different implementations may be combined in yet another implementation without departing from the disclosure or the recited claims.

1. An integrated on-chip system for molecular sequencing, the system comprising:
a semiconductor chip having integrated therein:
a main waveguide,
a plurality of branch waveguides optically connected to the main waveguide,
a plurality of nanochannels each having a fluid inlet and a fluid outlet,
a plurality of molecule traps, a molecule trap at an intersection of a branch waveguide and a nanochannel, and
a plurality of photodetectors operably connected to the plurality of molecule traps, one photodetector for a molecule trap, the photodetector to detect a spectral signature from a molecule in the molecule trap; and
a laser optically connected to the main waveguide.

2. The system of claim 1, wherein the system is for DNA sequencing, and the system further comprising a DNA polymerase in the molecule trap.

3. The system of claim 1, wherein the photodetectors are located proximate the molecule traps.

4. The system of claim 1, wherein the laser is integrated with the semiconductor chip.

5. The system of claim 1, wherein the laser is separable from the semiconductor chip.

6. The system of claim 1, further comprising a spectrometer integrated in the chip, the spectrometer comprising a plurality of filters and the plurality of photodetectors.

7. The system of claim 1, further comprising a computing system operably connected to the plurality of photodetectors to identify the spectral signature.

8. The system of claim 7, wherein the computing system is configured to identify wavelength or fluorescence and correlate it to the molecule in the trap.

9. The system of claim 8, wherein the computing system is configured to identify wavelength or fluorescence and correlate it to a nucleotide.

10. An integrated on-chip system for molecular sequencing, the system comprising:
a semiconductor chip having integrated therein:
a plurality of waveguides,
a plurality of nanochannels intersecting the plurality of waveguides,
a molecule trap at an intersection of a waveguide and a nanochannel, and
a photodetector operably connected to the molecule trap, the photodetector to detect a spectral signature from a molecule in the molecule trap;
a laser optically connected to the main waveguide; and
a computing system operably connected to the plurality of photodetectors to identify the spectral signature.

11. The system of claim 10, wherein the system is for DNA sequencing, the system further comprising a DNA polymerase in the molecule trap, and computing system configure to identify a nucleotide based on the spectral signature.

12. The system of claim 10, wherein the photodetector is located proximate the molecule trap.

13. The system of claim 10, wherein the laser is integrated with the semiconductor chip.

14. The system of claim 10, wherein the laser is separable from the semiconductor chip.

15. The system of claim 10, further comprising a spectrometer integrated in the chip, the spectrometer comprising a plurality of filters and the plurality of photodetectors.

16. A method of sequencing DNA in real-time, the method comprising:
providing a DNA single strand to a molecule trap at an intersection of a waveguide and a nanochannel, the waveguide and the nanochannel integrated in a semiconductor chip;
providing a light source from a laser to the molecule trap via the waveguide;
monitoring a spectral signature via a photodetector operably connected to the molecule trap, the spectral signature indicative of a free nucleotide attaching to the DNA single strand; and
correlating the spectral signature to a nucleotide.

17. The method of claim 16, wherein the free nucleotide is labeled and the spectral signature is fluorescence.

18. The method of claim 16, wherein the free nucleotide is non-labeled and the spectral signature is a wavelength or Raman signal.

19. The method of claim 16, wherein providing a light source is from a laser integrated in the semiconductor chip.

20. The method of claim 16, wherein providing a light source is from a laser removable and replaceable from the semiconductor chip.
